(19) United States
(12) Patent Application Publication
(10) Pub. No.: US 2003/0096808 A1
(43) Pub. Date: May 22, 2003

(54) SUBSTANCE TO PREVENT OR REVERSE WEIGHT GAIN INDUCED BY PSYCHOACTIVE AGENTS

(76) Inventor: JON M. MILLER, LOUISVILLE, KY (US)

Correspondence Address: DONALD L. COX, LYNCH, COX, GILMAN & MAHAN, AEGON CENTER- SUITE 2200, 400 W. MARKET, LOUISVILLE, KY, 40202, US

(21) Appl. No.: 09/280,279

(22) Filed: Mar. 29, 1999

(51) Int. Cl. A61K 31/551; A61K 31/5513; A61K 31/519; A61K 31/495; A61K 31/4439; A61K 31/19

(52) U.S. Cl. 514/220; 514/221; 514/259.41; 514/255.04; 514/341; 514/557

(57) ABSTRACT

A substance to prevent or reverse weight gain induced by psychoactive agents (10) having an antipsychotic drug (12) or mood stabilizing drug (14) in a concentration from 0.01% to 99.99% in combination with a histamine H2-receptor antagonist (16) in a concentration from 99.99% to 0.01%. The antipsychotic drug (12) is selected from a group consisting of olanzapine (12A), clozapine (12B), risperidone (12C), and quetiapine (12D). The antipsychotic drug (12) is typically in a concentration of 10% to 90%, 30% to 60% and 50%. The mood stabilizing drug (14) is selected from a group consisting of divalproex sodium (14A), valproic acid (14B), and mirtazapine (14C). The mood stabilizing drug (14) is typically in a concentration of 10% to 90%, 30% to 60% and 50%. The histamine H2-receptor antagonist (16) is selected from a group consisting of nizatidine (16A), famotidine (16B), cimetidine (16C) and ranitidine (16D). The histamine H2-receptor antagonist (16) is in a concentration of 90% to 10%. The histamine H2-receptor antagonist (16) is typically in a concentration of 60% to 30% and 50%.

### BACKGROUND OF THE INVENTION

1. Field of the Invention

The present invention relates to medications used for weight control. More particularly, the present invention relates to the use of a histamine H₂-receptor antagonist with antipsychotic and mood stabilizing drugs to control weight.

2. Description of the Prior Art

Numerous innovations for substances to prevent or reverse weight gain have been provided in the past. Even though these innovations may be suitable for the specific individual purposes to which they address, they differ from the present invention because they fail to describe or claim at least one combination of the features depicted in the present invention. Even though these innovations may be suitable for the specific individual purposes to which they address, they would not be suitable for the purposes of the present invention as heretofore described.

### SUMMARY OF THE INVENTION

The present invention prevents and reverses weight gain associated with the use of olanzapine and other antipsychotic drugs. The combination of psychoactive drugs and histamine H₂-receptor antagonists may represent a combined single dose delivery system or multiple drug regimen taken at preselected times. The psychoactive drugs are dosed as recommended by the manufacturer and the histamine H₂-receptor antagonists are dosed as for use in maintenance treatment of duodenal ulcer.

The types of problems encountered in the prior art are weight gain associated with the use of antipsychotic and mood stabilizing drugs, particularly olanzapine.

The problem was solved by the present invention because it was discovered that adding histamine H₂-receptor antagonists such as nizatidine or famotidine had a positive effect on weight gain associated with the use of antipsychotic and mood stabilizing drugs.

Accordingly, it is an object of the present invention to prevent or reduce weight gain in patients using antipsychotic and mood stabilizing medication.

In keeping with these objects, and with others which will become apparent hereinafter, one feature of the present invention resides, briefly stated, in the addition of histamine H₂-receptor antagonists to a regimen of psychoactive drugs such as the antipsychotic drugs, olanzapine, clozapine, risperidone, and quetiapine.

When the medication combination is designed in accordance with the present invention, weight gain is reduced or eliminated.

In accordance with another feature of the present invention, the addition of histamine H₂-receptor antagonists to a regimen of mood stabilizing drug such as divalproex sodium, valproic acid, and mirtazapine reduces or eliminates weight gain.

The novel features which are considered characteristic for the invention are set forth in the appended claims. The invention itself, however, both as to its construction and its method of operation, together with additional objects and advantages thereof, will be best understood from the following description of the specific embodiments when read and understood in connection with the accompanying drawings.

### LIST OF REFERENCE NUMERALS UTILIZED IN THE DRAWINGS

10—substance to prevent or reverse weight gain (10)

12—antipsychotic drug (12)

12A—olanzapine (12A)

12B—clozapine (12B)

12C—risperidone (12C)

12D—quetiapine (12D)

14—mood stabilizing drug (14)

14A—divalproex sodium (14A)

14B—valproic acid (14B)

14C—mirtazapine (14C)

16—histamine H2-receptor antagonist (16)

16A—nizatidine (16A)

16B—famotidine (16B)

16C—cimetidine (16C)

16D—ranitidine (16D)

### BRIEF DESCRIPTION OF THE DRAWINGS

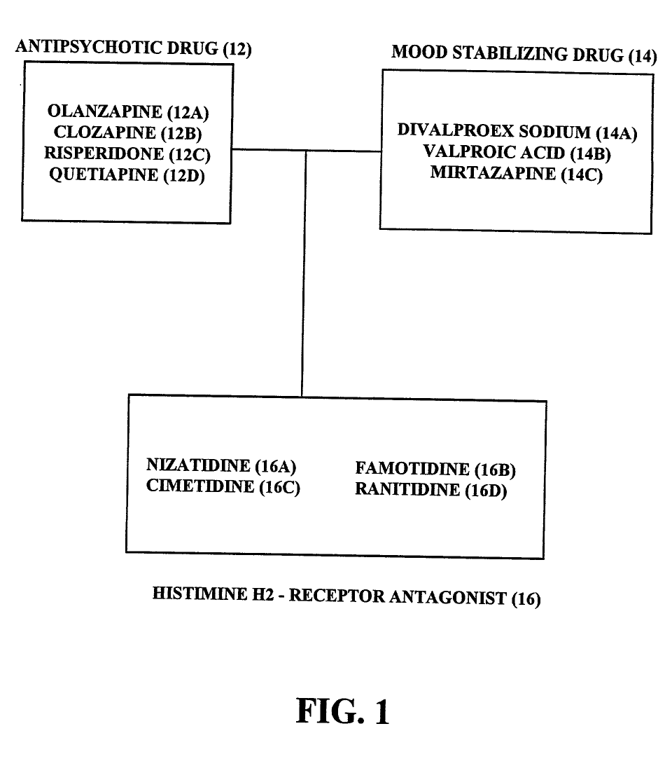

FIG. 1 is a block diagram of a substance to prevent or reverse weight gain.

### DESCRIPTION OF THE PREFERRED EMBODIMENT

Referring to FIG. 1 which is a block diagram of a substance to prevent or reverse weight gain. The substance to prevent or reverse weight gain (10) having an antipsychotic drug (12) or mood stabilizing drug (14) in a concentration from 0.01% to 99.99% in combination with a histamine H2-receptor antagonist (16) in a concentration from 99.99% to 0.01%.

The antipsychotic drug (12) is selected from a group consisting of olanzapine (12A), clozapine (12B), risperidone (12C), and quetiapine (12D). The antipsychotic drug (12) is typically in a concentration of 10% to 90%, 30% to 60% and 50%.

The mood stabilizing drug (14) is selected from a group consisting of divalproex sodium (14A), valproic acid (14B), and mirtazapine (14C). The mood stabilizing drug (14) is typically in a concentration of 10% to 90%, 30% to 60% and 50%.

The histamine H2-receptor antagonist (16) is selected from a group consisting of nizatidine (16A), famotidine (16B), cimetidine (16C) and ranitidine (16D). The histamine H2-receptor antagonist (16) is in a concentration of 90% to 10%. The histamine H2-receptor antagonist (16) is typically in a concentration of 60% to 30% and 50%.

The substance to prevent or reverse weight gain (10) is formulated in the following combinations;

10 parts of olanzapine (12A) are combined with 150 parts of nizatidine (16A) or ranitidine (16D)

10 parts of olanzapine (12A) are combined with 20 parts of famotidine (16B)

10 parts of olanzapine (12A) are combined with 400 parts of cimetidine (16C)

3 parts of risperidone (12C) are combined with 75 parts of nizatidine (16A) or ranitidine (16D)

3 parts of risperidone (12C) are combined with 10 parts of famotidine (16B)

3 parts of risperidone (12C) are combined with 200 parts of cimetidine (16C)

100 parts of quetiapine (12D) are combined with 50 parts of nizatidine (16A) or ranitidine (16D)

100 parts of quetiapine (12D) are combined with 7 parts of famotidine (16B)

100 parts of quetiapine (12D) are combined with 135 parts of cimetidine (16C)

30 parts of mirtazapine (14C) are combined with 150 parts of nizatidine (16A) or ranitidine (16D)

30 parts of mirtazapine (14C) are combined with 20 parts of famotidine (16B)

30 parts of mirtazapine (14C) are combined with 400 parts of cimetidine (16C)

250 parts of divalproex sodium (14A) are combined with 50 parts of nizatidine (16A) or ranitidine (16D)

250 parts of divalproex sodium (14A) are combined with 7 parts of famotidine (16B)

250 parts of divalproex sodium (14A) are combined with 135 parts of cimetidine (16C)

It will be understood that each of the elements described above, or two or more together, may also find a useful application in other types of constructions differing from the type described above.

While the invention has been illustrated and described as embodied in a Substance to Prevent or Reverse Weight Gain Induced by Psychoactive Agents, it is not intended to be limited to the details shown, since it will be understood that various omissions, modifications, substitutions and changes in the forms and details of the device illustrated and in its operation can be made by those skilled in the art without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

What is claimed is:

1. A substance to prevent or reverse weight gain induced by psychoactive agents (10) comprising:
A) an antipsychotic drug (12) or mood stabilizing drug (14) in a concentration from 0.01% to 99.99%; and
B) a histamine H2-receptor antagonist (16) in a concentration from 99.99% to 0.01%.

2. A substance to prevent or reverse weight gain induced by psychoactive agents (10) as described in claim 1, wherein the antipsychotic drug (12) is selected from a group consisting of olanzapine (12A), clozapine (12B), risperidone (12C), and quetiapine (12D).

3. A substance to prevent or reverse weight gain induced by psychoactive agents (10) as described in claim 1, wherein the mood stabilizing drug (14) is selected from a group consisting of divalproex sodium (14A), valproic acid (14B), and mirtazapine (14C).

4. The substance to prevent or reverse weight gain induced by psychoactive agents (10) as described in claim 1, wherein the histamine H2-receptor antagonist (16) is selected from a group consisting of nizatidine (16A),famotidine (16B), cimetidine (16C) and ranitidine (16D).

5. The substance to prevent or reverse weight gain (10) as described in claim 2, wherein the antipsychotic drug (12) is in a concentration of 10% to 90%.

6. The substance to prevent or reverse weight gain induced by psychoactive agents (10) as described in claim 5, wherein the antipsychotic drug (12) is in a concentration of 30% to 60%.

7. The substance to prevent or reverse weight gain induced by psychoactive agents (10) as described in claim 6, wherein the antipsychotic drug (12) is in a concentration of 50%.

8. The substance to prevent or reverse weight gain induced by psychoactive agents (10) as described in claim 3, wherein the mood stabilizing drug (14) is in a concentration of 10% to 90%.

9. The substance to prevent or reverse weight gain induced by psychoactive agents (10) as described in claim 8, wherein the the mood stabilizing drug (14) is in a concentration of 30% to 60%.

10. The substance to prevent or reverse weight gain induced by psychoactive agents (10) as described in claim 9, wherein the mood stabilizing drug (14) is in a concentration of 50%.

11. The substance to prevent or reverse weight gain induced by psychoactive agents (10) as described in claim 4, wherein the histamine H2-receptor antagonist (16) is in a concentration of 90% to 10%.

12. The substance to prevent or reverse weight gain induced by psychoactive agents (10) as described in claim 11, wherein the histamine H2-receptor antagonist (16) is in a concentration of 60% to 30%.

13. The substance to prevent or reverse weight gain induced by psychoactive agents (10) as described in claim 12, wherein the histamine H2-receptor antagonist (16) is in a concentration of 50%.

13. The substance to prevent or reverse weight gain induced by psychoactive agents (10) as described in claim 4, wherein 10 parts of olanzapine (12A) are combined with 150 parts of nizatidine (16A) or ranitidine (16D).

14. The substance to prevent or reverse weight gain induced by psychoactive agents (10) as described in claim 4, wherein 10 parts of olanzapine (12A) are combined with 20 parts of famotidine (16B).

15. The substance to prevent or reverse weight gain induced by psychoactive agents (10) as described in claim 4, wherein 10 parts of olanzapine (12A) are combined with 400 parts of cimetidine (16C).

16. The substance to prevent or reverse weight gain induced by psychoactive agents (10) as described in claim 4, wherein 3 parts of risperidone (12C) are combined with 75 parts of nizatidine (16A) or ranitidine (16D).

17. The substance to prevent or reverse weight gain induced by psychoactive agents (10) as described in claim 4, wherein 3 parts of risperidone (12C) are combined with 10 parts of famotidine (16B).

18. The substance to prevent or reverse weight gain induced by psychoactive agents (10) as described in claim 4, wherein 3 parts of risperidone (12C) are combined with 200 parts of cimetidine (16C).

19. The substance to prevent or reverse weight gain induced by psychoactive agents (10) as described in claim 4, wherein 100 parts of quetiapine (12D) are combined with 50 parts of nizatidine (16A) or ranitidine (16D).

20. The substance to prevent or reverse weight gain induced by psychoactive agents (10) as described in claim 4, wherein 100 parts of quetiapine (12D) are combined with 7 parts of famotidine (16B).

21. The substance to prevent or reverse weight gain induced by psychoactive agents (10) as described in claim 4, wherein 100 parts of quetiapine (12D) are combined with 135 parts of cimetidine (16C).

22. The substance to prevent or reverse weight gain induced by psychoactive agents (10) as described in claim 4, wherein 30 parts of mirtazapine (14C) are combined with 150 parts of nizatidine (16A) or ranitidine (16D).

23. The substance to prevent or reverse weight gain induced by psychoactive agents (10) as described in claim 4, wherein 30 parts of mirtazapine (14C) are combined with 20 parts of famotidine (16B).

24. The substance to prevent or reverse weight gain induced by psychoactive agents (10) as described in claim 4, wherein 30 parts of mirtazapine (14C) are combined with 400 parts of cimetidine (16C).

25. The substance to prevent or reverse weight gain induced by psychoactive agents (10) as described in claim 4, wherein 250 parts of divalproex sodium (14A) are combined with 50 parts of nizatidine (16A) or ranitidine (16D).

26. The substance to prevent or reverse weight gain induced by psychoactive agents (10) as described in claim 4, wherein 250 parts of divalproex sodium (14A) are combined with 7 parts of famnotidine (16B).

27. The substance to prevent or reverse weight gain induced by psychoactive agents (10) as described in claim 4, wherein 250 parts of divalproex sodium (14A) are combined with 135 parts of cimetidine (16C).
